(19) United States
(12) Patent Application Publication
(10) Pub. No.: US 2002/0136782 A1
(43) Pub. Date: Sep. 26, 2002

(54) Composition patent for solid-dosage form of weight loss product

(76) Inventor: Albert M. Fleischner, Cedar Knolls, NJ (US)

Correspondence Address: MARK POHL, 55 MADISON AVENUE, 4TH FLOOR, MORRISTOWN, NJ, 07960

(21) Appl. No.: 09/761,622

(22) Filed: Jan. 18, 2001

(51) Int. Cl. A61K 35/78; A61K 33/32; A61K 33/24

(52) U.S. Cl. 424/725; 424/774; 424/641; 424/655

(57) ABSTRACT

Supplement compositions designed to support weight loss and increase energy.

### BACKGROUND

The prior art regarding this invention arises from distinct areas not heretofore combined to create new and useful formula sets or new and useful improvements thereof regarding a Solid-dosage Form of a Weight Loss Product.

This invention relates to the evolving science that a new and unique combination of ma huang extract (ephedrine alkaloids), caffeine and glucosamine sulfate results in increased weight loss and energy.

Ma huang is a central nervous system stimulant that has the ability to open up the adrenergic receptor sites found primarily in the heart and lungs, thereby increasing the metabolic rate and calorie expenditure. The net result is the release of fatty acids from stored fat cells and a quicker conversion of the fat into energy. When combined with a modest amount of caffeine the thermogenic effects can be improved as much as 20%¹. Ma huang also acts as an appetite suppressant.²

A double-blind, placebo-controlled study published in Metabolism finds a thermogenic synergism between ephedrine and caffeine.³ A second study published in the International Journal of Obesity Related Metabolic Disorders concludes that the ephedrine/caffeine combination is safe and effective in long-term treatment in improving and maintaining weight loss.⁴ A third study, published in the American Journal of Clinical Nutrition, concludes that results show that ephedrine and caffeine can promote weight loss through an increase in energy expenditure or, in some individuals, a combination of an increase in energy expenditure and a decrease in food intake.⁵

In addition, the ephedrine/caffeine combination has lean body mass saving properties ^{6, 7} and abolishes the decline in HDL-cholesterol during active weight loss due to the beta-agonistic properties of ephedrine.⁸ Due to increased central nervous system stimulation, the combination significantly prolongs exercise time to exhaustion and improves performance in runners.^{9, 10}

Glucosamine, in the form of glucosamine sulfate, another ingredient of this compound, also contributes to weight loss. When food intake by the body occurs at a faster rate than energy consumption, the cellular concentration of adenosine triphosphate rises. Cells, however, do not store extra energy in the form of extra adenosine triphosphate. When cellular adenosine triphosphate concentrations rise because more energy (from food) is available than can be immediately used, high adenosine triphosphate concentrations inhibit glycolysis. Under conditions of high cellular adenosine triphosphate concentrations, when glycolysis is inhibited, glucose is instead converted into glycogen and fat.

When fat stored in adipose tissue is going to be used as an energy source, lipase enzymes hydrolyze triglycerides into glycerol and free fatty acids in a process called lipolysis (the breakdown of fat). These molecules (primarily the free fatty acids) serve as blood-borne energy carriers that can be used by the liver, skeletal muscles, and other organs for aerobic respiration.¹¹

The effect of insulin on lipogenesis, the formation of fatty acids in the body, is blocked by glucosamine, indicating that glucosamine plays a role as a messenger for this insulin effect.¹² Insulin is secreted when there is high sugar content; insulin secretion allows for fat storage. High insulin levels trigger the hypothalamus to send hunger signals, which sets off a craving for carbohydrates; this leads one to eat more, which leads to more insulin. Excess carbohydrates are converted into glucose, and then stored as fat.

Glucose triggers a rise in insulin. Insulin acts to lower blood glucose levels, regulating those levels through several actions, including lipogenesis (conversion of carbohydrate and protein into fat). Fat cells can't be metabolized when insulin levels are normal. When you have high insulin levels, you block lipolysis and store fat. The body must initiate lipolysis to supply the cellular energy source ATP, which is necessary for muscle contraction (energy). When you reduce the level of insulin, you burn fat to provide energy. Glucosamine blocks the effect of insulin, burning up stored fat and resulting in weight loss.¹³

Scientists have yet to determine at what point in the metabolic pathway glucosamine acts to block insulin, but several studies have demonstrated this fact. A study at the Washington University School of Medicine concluded that direct administration of glucosamine can rapidly lower cellular ATP levels and affect insulin action in fat cells by independent mechanisms.¹⁴ A University of Southern California study further explains that glucosamine induced complete and reversible insulin resistances¹⁵. A third study at the Albert Einstein College of Medicine adds that the etiology of peripheral insulin resistance may be distinct from the rapid and marked impairment in insulin signaling and that glucosamine on insulin-stimulated glucose metabolism is a different mechanism.¹⁶ Essentially, the glucosamine keeps the glucose from being stored as fat, providing a temporary and reversible hyperglycemic effect that allows the glucose to be used as energy instead by blunting the insulin-induced increase in muscle glycogen content.¹⁷ In addition to the hyperglycemic effect of glucosamine, Japanese studies on dogs and ducks demonstrate that glucosamine causes glucagon release in addition to its effect to suppress insulin release as well as its direct inhibitory effect on glucose utilization in tissues.¹⁸

By increasing the metabolic rate and calorie expenditure with ma huang and caffeine while simultaneously encouraging the body to use stored fat for energy, in conjunction with the carefully blended composition of other ingredients, this new product provides a unique and successful method for losing weight and increasing energy.

Curriculum Vitae

Albert M. Fleischner, Ph.D., has a doctorate in Pharmaceutical Chemistry from Rutgers University and has had over thirty years experience in the pharmaceutical industry with firms such as Schering Corporation, Lehn & Fink Division of Sterling Drugs, Bradley Pharmaceutical Corporation, Amerchol Division of CPC and the Goen Group companies. He has a number of published papers and two previously granted patents and has several patents pending.

### SUMMARY

The invention discloses the formula sets that embody the invention of the supplement composition for increasing weight loss and energy levels. The combination of ephedrine and caffeine increases fat loss, maintains muscle mass, prevents the fall of HDL cholesterol during weight loss, increases insulin sensitivity, reduces lipogenesis and is safe. With the addition of glucosamine sulfate, the new and useful formula is further enhanced.

We now discuss in detail the most preferred version, variants or embodiments of the invention. First, a few words on terminology. The claim term “a” includes one and more than one. The claim term “label” is used as defined in the Federal Food Drug & Cosmetic Act and the regulations promulgated thereunder. We now turn to discussing in great detail the best (or “preferred”) versions (or “embodiments”) of the invention.

A representative formula for Solid-Dosage Form of Weight Loss Product is as follows, one tablet contains:

| | |
|---|---|
| Vitamin B₆ (as pyridoxine HCl) | 3.4 mg |
| Zinc (as zinc citrate and arginate) | 2.5 mg |
| Manganese (as manganese arginate) | 1.0 mg |
| Chromium (as chromium Chelavite ™ | 100.0 mcg |
| dinocitinate glycinate)† | |
| Gymnema sylvestre leaf and Gymnema sylvestre | 83.4 |
| leaf extract (25% gymnemic acids) | |
| Vanadium | 34.5 mg |
| Glucosamine sulfate | 100.0 mg |
| Lecithin | 50.0 mg |
| Inositol | 13.4 mg |
| DL-methionine | 10.0 mg |
| Choline bitartrate | 10.08 mg |
| Peppermint leaf | 12.6 mg |
| Fennel seed | 8.0 mg |
| Bladderwrack kelp | 5.0 mg |
| L-glutamine | 2.0 mg |
| DL-phenylalanine | 2.0 mg |
| Rosemary leaf | 2.0 mg |
| L-Tyrosine | 2.0 mg |
| Bitter melon extract | 0.5 mg |
| Guarana seed extract (40 mg caffeine) | 192.0 (20.0-50.0 mg |
| | caffeine) |
| Ma huang extract (12 mg ephedrine alkaloids) | 150.0 mg (5.0-28.0 |
| | mg (aerial |
| | parts) ephedrine |
| | alkaloids) |
| Black pepper | 13.7 mg |
| Ginger root | 15.1 mg |
| Long pepper | 13.7 mg |
| Spirulina blue-green algae | 3.0 mg |
| Cayenne pepper | 2.0 mg |
| Kola nut | 2.0 mg |
| Siberian ginseng root | 2.0 mg |
| Cinnamon twig | 1.7 mg |
| Lemon verbena herb | 1.7 mg |
| Chamomile flower | 1.4 mg |
| Licorice root | 0.7 mg |
| Tangerine peel | 0.7 mg |
| Chicory root | 0.3 mg |
| Excipients: | |
| Dicalcium Phosphate, Microcrystalline Cellulose, | a sufficient quantity |
| Croscarmellose Sodium, Stearic Acid, Magne- | of each to make a |
| sium Stearate, Silica, Pharmaceutical Glaze | suitable tablet |

The scientific rationale for the formulation is as follows:

Vitamin B6 (pyridoxine) helps the body process the protein, fat and carbohydrates in our diet. It is required in the metabolization of carbohydrate, fats and proteins and has a primary role in the utilization of proteins and amino acids, converting them to carbohydrates or fats for storage or energy. Vitamin B₆ also helps the body resist stress. Vitamin B₆ activates the release of glucogen from the muscles and liver and is thus responsible for the production of biological energy. It works with other vitamins and minerals to supply the energy used in our muscles, and plays a role in cell growth.¹⁹

Zinc arginate is a more absorbable form of zinc. Zinc citrate is well tolerated by most individuals and contains citric acid, an antioxidant. A positive correlation between zinc and leptin has been recently noted, and both are known as important mediators in appetite control. A recent study indicates that the amelioration of sucrose-induced obesity by zinc repletion may be partly attributable to the hyperleptinemia induced by the mineral.²⁰

Zinc also helps red blood cells pick up carbon dioxide and drop it off in the lungs to be exhaled. This exchange helps maintain the chemical environment muscle cells need to contract and produce energy.²¹ Zinc deficiency acts as a sustaining factor in abnormal eating behavior. It's known that zinc activates the areas of the brain that govern taste and smell, and without those the desire to eat is vastly diminished.

Manganese is a mineral that is required to manufacture enzymes necessary for the metabolism of proteins and fats, including those involved in blood sugar control, thyroid function, and energy metabolism.²²

A deficiency in chromium results in glucose intolerance. Chromium contributes to the prevention of adult-onset diabetes. It has been shown to decrease sugar cravings and is considered an effective treatment against both hypoglycemia and diabetes by improving glucose tolerance, increasing cell sensitivity to insulin, and reducing circulating insulin levels.²³

Research also indicates chromium's role in lowering total cholesterol, LDL cholesterol, and serum triglyceride levels and improves the LDL-to-HDL cholesterol ratio, according to Dr. Jeoffry Gordon in San Diego, Calif. Subsequent research supports this and suggests a greater role for chromium in the treatment and prevention of high cholesterol and cardiovascular disease. There is speculation chromium positively affects lipid profiles by its ability to increase insulin efficiency, thereby reducing elevated lipid levels.²⁴

Chromium polynicotinate has been shown to possess greater biological activity and is safer than other chromium supplements. It potentiates the effects of insulin and helps overcome insulin resistance in overweight people. Chromium also seems to stimulate thermogenesis, the burning of fat, without any physical exertion.²⁵

Chromium picolinate was found to cause significant chromosome damage at a non-toxic dose. In contrast, chromium polynicotinate did not cause chromosome damage at equivalent doses. Consumers are urged to switch to a nontoxic form of chromium, such as niacin-bound chromium, also known as chromium polynicotinate.²⁶ A 1998 study demonstrated that 9 out of 10 American diets fall short in chromium. The chromium levels of more than 40,000 men and women were measured in this study. After comparing various age groups, the study found that chromium levels plummet with the passage of time.²⁷ According to Dr. Michelle Rubin from the University of Maryland, in addition to increasing as we age, chromium needs rise dramatically during exercise.²⁸

The gradual hypoglycemic (blood sugar-lowering) action of gymnema leaves was first documented in the late 1920s.²⁹ Gymnema leaves raise insulin levels in healthy volunteers.³⁰ This may be due to regeneration of the cells in the pancreas that secrete insulin.³¹ Animal research shows that gymnema can also improve uptake of glucose into cells and prevent adrenaline from stimulating the liver to produce glucose, thereby raising blood sugar levels.³² The leaves are also noted for lowering serum cholesterol and triglycerides. ³³ Gurmarin, another constituent of the leaves, and gymnemic acid have been shown to block the ability to taste sweets in humans.³⁴

Vanadium is needed for cellular metabolism and for thyroid function. Research at the Grand Forks Human Nutrition Research Center suggests that vanadium is an essential nutrient beneficial for thyroid hormone metabolism.³⁵ A study at the University of British Columbia found that vanadium compounds could correct defective signaling pathways and increase the cells' response to insulin, which would aid normal processing of sugar in patients with diabetes.³⁶ Vanadium can mimic insulin. In other words, in research done with cells, it has been able to replace insulin.³⁷ Vanadium has recently been observed to have several physiological insulin-like effects by a post-insulin receptor kinase mechanism, making it very likely to have a favorable effect on carbohydrate metabolism.³⁸

The benefits of glucosamine have already been discussed in detail, above.

There is evidence that lecithins possess a lipotropic action, i.e., they correct or check abnormalities in lipid metabolism. They will, for example, prevent the fatty livers that generally result when depancreatized animals are maintained with insulin and fed diets high in carbohydrates. A lipotropic agent may be defined as one which is involved in the conversion of neutral fats to phospholipids, a form in which they are more readily transported in the bloodstream. Choline is perhaps the single most effective such agent and is a part of the normally functioning metabolic cycle of fat. The lack of normal ability to metabolize fat leads to a consistently higher than normal serum lipid level and a less rapid return of the serum lipid level to the previous level following ingestion of fat. It has been observed that the most common specific lipotropic agents (choline, inositol and methionine) are effective when administered singly but, in addition, appear to have a synergistic action when administered simultaneously.³⁹

Inositol is required for proper formation of cell membranes and helps in transporting fats within the body.⁴⁰ Methionine is one of the nine essential amino acids; its major role is to facilitate fat and protein metabolism. It supplies sulfur and other compounds required by the body for normal metabolism and growth.⁴¹

Peppermint is the first of the ingredients that are part of the Appetite Control Blend. Peppermint is used as a carminative, allowing gas pressure to escape the stomach. It aids digestion and is held to be helpful for many stomach problems, irritable bowel syndrome, nausea, morning sickness, diarrhea, constipation and flatulence.⁴² Peppermint enhances digestion by increasing stomach acidity. It slightly anesthetizes mucous membranes and the gastrointestinal tract.⁴³

Fennel seed promotes gastrointestinal motility and, in higher concentrations, acts as an antispasmodic.⁴⁴ Bladderwrack kelp is one of the richest natural sources of approximately 30 trace elements and major minerals. It regulates the thyroid function and may be very helpful in reducing obesity where it is associated with thyroid trouble. Bladderwrack kelp is also a metabolic stimulant.⁴⁵

At least three constituents of bitter melon have been reported to have hypoglycemic (blood-lowering) actions. Multiple controlled clinical studies have confirmed the benefit of bitter melon for people with diabetes.⁴⁶ Evidence indicates it works by stimulating insulin release from the pancreas.⁴⁷ Bitter melon is also thought to stimulate digestive function.⁴⁸

Rosemary leaf relaxes the stomach and stimulates circulation and digestion.⁴⁹ Rosemary can serve as a source of natural antioxidants.⁵⁰

L-Glutamine is one of the most common and widely used amino acids in the body. It is a carbon and nitrogen donor and helps replenish glycogen, which restores energy. In fact, it is used as a source of energy and nucleotide synthesis for all rapidly dividing cells in the body. In the absence of glucose, the body will use glutamine for energy, thus preventing hypoglycemia without muscle loss.⁵¹

Phenylalanine is one of two amino acids that govern the release of an intestinal hormone called cholecystokinin, known as CKK. This hormone signals the brain to feel satisfied after eating. People given CCK stop eating and feel full sooner. L-phenylalanine has a nutritional value; D-phenylalanine has painkilling and depression alleviating properties. DL-phenylalanine is a 50/50 mixture of these two forms. Its activity is enhanced by the presence of Vitamin B₆.⁵²

The body uses phenylalanine to manufacture tyrosine, which is the precursor to the neurotransmitter dopamine. Dopamine controls desire in the brain. It also has anti-depressive qualities.⁵³ Tyrosine produces thyroxin, which plays an important role in controlling metabolic rate. It may also be used as a mild appetite suppressant. It was proven in research studies that tyrosine supplementation results in increased performance over a control group.⁵⁴

Guarana seed extract (providing 40 mg of caffeine) is the first of the ingredients in the Thermogenic Herbal Concentrates group. Caffeine and the closely related alkaloids theobromine and theophylline make up the primary active agents in guarana. Caffeine 's effects are well known and include stimulating the central nervous system, increasing metabolic rate and having a mild diuretic effect.⁵⁵ The tannic acid in guarana is astringent and probably accounts for its use as a digestive tonic. It is also a stimulant.⁵⁶

The benefits of ma huang extract and its ephedrine alkaloids have been discussed above.

Black pepper stimulates the production of gastric juices (carminative), aids digestion, strengthens the stomach, and tones the spleen.⁵⁷ It has also been reported to have strong lipolytic activity that resides in the outer layer of the fruit.⁵⁸ It has also been reported to have antioxidant properties.

Ginger is said to stimulate gastric juices and has carminative properties.⁵⁹ Ginger contains gingerol, an oleoresin that accounts for the characteristic aroma of ginger and explains its therapeutic properties. Components of gingerol have recently been studied and found to possess beneficial properties for the treatment of poor digestion, heartburn, vomiting and motion sickness.⁶⁰

Long pepper is thermogenic, carminative, expectorant and aids digestion.⁶¹ Spirulina, a type of algae, is a rich source of protein, vitamins, minerals and essential fatty acids. In one double-blind study of sixteen overweight individuals, ingestion of 2.8 grams of spirulina three times per day for four weeks resulted in a small but statistically significant weight loss.⁶²

Cayenne causes the brain to secrete more endorphins. It is considered thermogenic and is rich in Vitamin C.⁶³ It is said that a poorly nourished pituitary gland is sometimes responsible for people being overweight; cayenne also aids the pituitary gland to release an ample supply of protein, which helps adjust hormone balance in the body.⁶⁴

Kola nut diminishes sensations of hunger and fatigue. It also acts as an aid to digestion.⁶⁵ The properties of kola are the same as caffeine, modified only by the astringents present. Fresh kola nuts have stimulant action apart from the caffeine content. The active ingredients work together to stimulate the central nervous system, act as an anti-depressive and relieve fatigue and hunger.⁶⁶

Ginseng strengthens the adrenal and reproductive glands and is useful for lack of energy, due to a general stimulatory effect that raises the physical and mental capacity for work.^{67, 68} Results of a study published in the American Journal of Clinical Medicine show that ginseng at appropriate doses improves learning, memory and physical capabilities. A Canadian study shows that ginseng saponins enhance exercise endurance by altering fuel homeostasis during prolonged exercise, presumably by increasing FFA utilization in preference over glucose for cellular energy demands.⁶⁹

Lemon verbena is a febrifuge and sedative. It is used as a stomachic, antipyretic and antispasmodic in dyspepsia, indigestion and flatulence.⁷⁰ Lemon verbena also has anti-inflammatory activity.⁷¹

Cinnamon has compounds with insulin-potentiating activity, thus lowering the amount of insulin required for glucose metabolism.⁷² Cinnamon is used as a carminative, astringent, antiseptic and as a stimulant. It stops vomiting and relieves flatulence.⁷³ Cinnamon also reportedly has strong lipolytic properties.⁷⁴

Chamomile is an anti-inflammatory, as well as a traditional remedy for stress and anxiety, indigestion and insomnia.⁷⁵ Chamomile produces a brilliant blue oil containing the compound azulene that is thought to serve as a natural digestive aid.⁷⁶

Tangerine peel is useful for digestive disorders. It promotes proper digestion and relieves nausea and gas.⁷⁷

Licorice is an anti-inflammatory. Its sweet flavor masks other, medicinal flavors.⁷⁸ Licorice cleanses the colon, decreases muscular spasms and promotes adrenal gland function.⁷⁹

Chicory is a tonic, laxative and diuretic.⁸⁰ It is used for dyspeptic disorders.

Without further elaboration, it is believed that one skilled in the art can, using the preceding description, utilize the present invention to its fullest extent. The specific formulas are included as a preferred embodiment of the composition formula ranges, and not to further qualify the description. Claim references to specific components include the component itself, as well as concentrates, metabolites, constituents, extracts or combinations of said ingredients.

### FOOTNOTES

¹ http://www.ephedra.net

² Leung, A., Foster, S. Encyclopedia of Common Natural Ingredients Used in Food, Drugs, and Cosmetics. 1996;2:228.

³ Metabolism 1991;March;40(3):323-9.

⁴ Int J Obes Relat Metab Disord 1993 December;17 Suppl 3:573-7;discussion 582.

⁵ Am J Clin Nutr 1998 July;68(1):42-51.

⁶ Int J Obes Relat Metab Disord 1993 February;17 Suppl 1:569-72.

⁷ Metabolism 1992 July;41(7):686-8.

⁸ Int J Obes Relat Metab Disord 1994 May;18(5):329-32.

⁹ Eur J Appl Physiol 1998 April;77(5):427-33.

¹⁰ Bell, D G, Jacobs, I. Aviat Space Environ Med 1999; 70:325-9.

¹¹ Fox, Stuart I. Human Physiology, 5^{th} ed., p. 112.

¹² Biochem J 1990 March 15;266(3):909-16.

¹³ ibid.

¹⁴ J Biol Chem 1998 August 7;273(32):20658-68.

¹⁵ Am J Physiol Endocrinol Metab 2000 January;278(1):E103-12.

¹⁶ J Biol Chem 1999 October 29;274(44):31312-9.

¹⁷ Diabetes 1999 August;48(8):1562-71.

¹⁸ Endocrinol Jpn 1975 December;22(6):517-23.

¹⁹ http://health.excite.com/content/article/3187.10144 Excite Health with WebMD

²⁰ Mann, Denise Zinc Deficiency Linked to Weight Loss Hormone Medical Tribune News Service 1998; June 29.

²¹ http://www.ars.usda.gov/is/pr/1999/990817.htm.

²² http://www.bodywise.com/prdoucts/ingredients/manganese.htm

²³ http://www.vitaminsplus.com/library/minerals/chromium.asp

²⁴ Ibid.

²⁵ http://members.aol.com/gwag16/dietnutrition/whatis.htm

²⁶ http://anndeweesallen.com/dal ra01.htm

²⁷ Dolby, Victoria. Chromium at the Energy Crossroads. Better Nutrition Magazine, 1998;May

²⁸ http://qualitycounts.com/fp/chromiumat.html

²⁹ Mhasker K S, Caius J F. A study of Indian medicinal plants. II. Gymnema sylvestre R.Br. Indian J Med Res Memoirs 1930;16:2-75.

³⁰ Shanmugasundaram K R, et al. Insulinotropic activity of G. sylvestre, R.Br. and Indian medicinal herb used in controlling diabetes mellitus. Pharmacol Res Commun 1981;13:475-86.

³¹ Shanmugasundaram E R, et al. Possible regeneration of the islets of Langerhans in streptozotocin diabetic rats given Gymnema sylvestre leaf extracts. J Ethnopharmacol 1990;30:265-79.

³² Gymnema sylvestre. Alt Med Rev 1999;4:46-7[review].

³³ Bishayee A, Chatterjee M. Hypolipidemic and antiatherosclerotic effects of oral Gymnema sylvestre R.Br. leaf extract in albino rats fed on a high fat diet. Phytother Res b 1994;8:118-20.

³⁴ http://www.healthwell.com/healthnotes/Herb/Gymnema.cfm

³⁵ http://www.gfhnrc.ars.usda.gov/News/nws9808a.htm

³⁶ http://www.publicaffairs.ubc.ca/mr/mr98/mr-98- 100.html

³⁷ http://www.diabetesnet.com/vanad.html

³⁸ http://www.mdschoice.com/text/abstracts/vanadiab.htm Role of Essential Trace Elements in the Disturbance of Carbohydrate Metabolism

³⁹ Textbook of Organic Medicinal and Pharmaceutical Chemistry Wilson C, Gisvold O-3^{rd} ed. 1956. p.39.

⁴⁰ http://www.mothernature.com/ency/Supp/inositol.asp

⁴¹ http://www.mothernature.com/ency/Supp/Methionine.asp

⁴² http://64.224.111.158/IMCAccess/ProfHerbs/Peppermintph.html

⁴³ Balch, James F. Prescription for Nutritional Healing: A Practical A to Z Reference to Drug-Free Remedies Using Vitamins, Minerals, Herbs & Food Supplements James F. Balch, Phyllis A. Balch-2^{nd} ed. 1977. p.75.

⁴⁴ http://onhealth.webmd.com/alternative/resource/herbs/item,15995.asp

⁴⁵ http://seasilver.threadnet.com/Preventorium/kelp.htm

⁴⁶ http://www.healthwell.com/healthnotes/Herb/Bitter Melon.cfm

⁴⁷ http://thp.sickbay.com/substance.asp?ID=21

⁴⁸ http://www.healthwell.com/healthnotes/Herb/Bitter Melon.cfm

⁴⁹ Balch, James F. Prescription for Nutritional Healing: A Practical A to Z Reference to Drug-Free Remedies Using Vitamins, Minerals, Herbs & Food Supplements James F. Balch, Phyllis A. Balch-2^{nd} ed. 1977. p.76.

⁵⁰ Tyler, V. E., Brady L.R. Pharmacognosy, 8^{th} ed. p. 76.

⁵¹ http://www.naturalhealthconsult.com/Monographs/glutamine.html

⁵² http://www.smartbasic.com/glos.aminos/phenylalanine.glos.html

⁵³ http://www.cockatoo.com/damiana/aminoacids.htm

⁵⁴ http://www.lef.org/protocols/prtcls-txt/t-prtc189.html

⁵⁵ Leung A Y, Foster S. Encyclopedia of Common Natural Ingredients Used in Food, Drugs, and Cosmetics, 2d ed. New York: John Wiley & Sons, 1996, 293-4.

⁵⁶ http://www.health-pages.com/gu/

⁵⁷ http://www.imm.org.pl/bird/pepper.htm

⁵⁸ Tyler, V. E., Brady L. R. Pharmacognosy, 8^{th} ed. p. 416.

⁵⁹ Ibid, p. 272.

⁶⁰ http://www.hometownrx.com/Docs/Herbs/Ginger.html

⁶¹ http://www.healthseva.com/content/home remedies/herbs/indianlong.php3

⁶² http:/www.mothernature.com/ency/concern/Weight Loss.asp

⁶³ http://www.herba-medica.com/remedies/capsicum.html

⁶⁴ Ibid.

⁶⁵ http://www.britannica.com/bcom/eb/article/3/0,5716,46993+1+45921,00.html?query=kola%20nut

⁶⁶ http://www.nopal.com/kolanut.html

⁶⁷ Balch, James F. Prescription for Nutritional Healing: A Practical A to Z Reference to Drug-Free Remedies Using Vitamins, Minerals, Herbs & Food Supplements James F. Balch, Phyllis A. Balch-2^{nd} ed. 1977. p.71.

⁶⁸ Petkov V D, Mosharrof A H, Effects of standardized ginseng extract on learning, memory and physical capabilities. Am J Chin Med 1987;15(1-2):19-29.

⁶⁹ Wang L C, Lee T F, Effect of ginseng saponins on exercise performance in non-trained rats. Planta Med 1998 March;64(2):130-3.

⁷⁰ http://www.hort.purdue.edu/newcrop/med-aro/factsheets/LEMON VERBENA.html

⁷¹ Deepak M, Handa S S Anti-inflammatory activity and chemical composition of extracts of verbena officinalis. Phytother Res 2000 September;14(6):463-5.

⁷² http:/ott.arsusda.gov/Inv/a395565.htm

⁷³ www.botanical.com/botanical/mgmn/c/cinnam69.html

⁷⁴ Tyler, V. E., Brady L. R. Pharmacognosy, 8^{th} ed. p. 168

⁷⁵ Balch, James F. Prescription for Nutritional Healing: A Practical A to Z Reference to Drug-Free Remedies Using Vitamins, Minerals, Herbs & Food Supplements James F. Balch, Phyllis A. Balch-2^{nd} ed. 1977. p.67

⁷⁶ http:www.lifehealthenergy.com/htdocs/TheMall/herbs/chamomile.htm.

⁷⁷ Powerful and Unusual Herbs from the Amazon and China, the World Preservation Society, Inc. 1993, 1995.

⁷⁸ Tyler, V. E., Brady L. R. Pharmacognosy, 8^{th} ed. p. 348.

⁷⁹ http://www.rain-tree.com/licorice.htm

⁸⁰ http://www.botanical.com/botanical/mgmn/c/chicor61.html

I claim:

1. A composition of matter intended to support weight loss and increase energy,
(a) said composition of matter containing (i) Vitamin B₆ and (ii) zinc, and (iii) (iv) manganese, and (v) chromium, and (vi) gymnema sylvestre leaf and extract, and (vii) vanadium, and (viii) glucosamine sulfate, and (ix) lipotropic blend, and (x) appetite control blend, and (xi) thermogenic herbal concentrates.
(b) said composition of matter intended for ingestion in capsule or tablet form; and
(c) said composition of matter not represented for use as a conventional food or as the sole item of a meal or diet; and
(d) said composition of matter labeled as a supplement for use in or by humans.

2. A composition of matter intended to support weight loss and increased energy.

3. (a) said combination of matter containing (i) ma huang extract and (ii) guarana, and (iii) glucosamine sulfate, and (iv) additional dietary substances which support the primary ingredients' activities.
(b) same
(c) same
(d) same

4. The dietary supplement of claim #3 wherein said ingredients are present in any combination thereof in the following approximate amounts:

| | |
|---|---|
| Vitamin B₆ (as pyridoxine HCl) | 1.0-50.0 g |
| Zinc (as zinc citrate and arginate) | 2.5-50.0 mg |
| Manganese (as manganese arginate) | 1.0-5.0 mg |
| Chromium (as chromium Chelavite ™ | 50.0-200.0 mcg |
| dinocitinate glycinate)† | |
| Gymnema sylvestre leaf and Gymnema sylvestre | 50.0-150.0 mg |
| leaf extract (25% gymnemic acids) | |
| Vanadium | 10.0-50.0 mg |
| Glucosamine sulfate | 50.0-500.0 mg |
| Lecithin | 25.0-100.0 mg |
| Inositol | 5.0-35.0 mg |
| DL-methionine | 5.0-25.0 mg |
| Choline bitartrate | 5.0-50.0 mg |
| Peppermint leaf | 5.0-20.0 mg |
| Fennel seed | 2.0-15.0 mg |
| Bladderwrack kelp | 1.0-100.0 mg |
| L-glutamine | 1.0-5.0 mg |
| DL-phenylalanine | 1.0-50.0 mg |
| Rosemary leaf | 1.0-10.0 mg |
| L-Tyrosine | 1.0-10.0 mg |
| Bitter melon extract | 0.5-2.0 mg |
| Guarana seed extract (40 mg caffeine) | 20.0-50.0 mg caffeine |
| Ma huang extract (12 mg ephedrine alkaloids) | 5.0-28.0 mg |
| (aerial parts) | ephedrine alkaloids |
| Black pepper | 5.0-15.0 mg |
| Ginger root | 5.0-50.0 mg |
| Long pepper | 5.0-15.0 mg |
| Spirulina blue-green algae | 1.0-10.0 mg |
| Cayenne pepper | 0-2.0 mg |
| Kola nut | 1.0-5.0 mg |
| Siberian ginseng root | 1.0-5.0 mg |
| Cinnamon twig | 1.0-5.0 mg |
| Lemon verbena herb | 1.0-5.0 mg |
| Chamomile flower | 1.0-5.0 mg |
| Licorice root | 0-10.0 mg |
| Tangerine peel | 0-10.0 mg |
| Chicory root | 0-10.0 mg |
| Excipients: | |
| Dicalcium Phosphate, Microcrystalline Cellulose, | a suitable amount of |
| Croscarmellose Sodium, Stearic Acid, Magne- | each to make a |
| sium Stearate, Silica, Pharmaceutical Glaze | tablet or capsule |

†Chelavite is a trademark of Albion Laboratories and is protected by U.S. Pat. Nos. 4,830,716; 4,599,152; and 4,167,564.
